(19) United States
(12) Patent Application Publication
(10) Pub. No.: US 2023/0366527 A1
(43) Pub. Date: Nov. 16, 2023

(54) CANDLE WARMER LAMP

(71) Applicant: Guangzhou Yona Import and Export Co., Ltd., Guangzhou (CN)

(72) Inventor: JINGEN HE, Guangzhou (CN)

(21) Appl. No.: 18/351,677

(22) Filed: Jul. 13, 2023

(51) Int. Cl. F21V 19/04; F21S 13/12; F21S 6/00; F21V 23/00; F21V 35/00

(52) U.S. Cl. CPC F21V 19/04; F21S 13/12; F21S 6/002; F21V 23/008; F21V 23/001; F21V 35/00

(57) ABSTRACT

The present disclosure provides a candle warmer lamp. The candle warmer lamp includes a heating device, a base, and a supporting part. The heating device is connected to the base through the supporting part, and the heating device is located above the base. The heating device includes a lamp holder and a heating lamp head, the lamp holder and a heating lamp head are detachably connected.

### TECHNICAL FIELD

The present disclosure belongs to heating lamps technology, in particular to a candle warmer lamp.

### BACKGROUND

With the improvement of people's living standards, people tend to use electronic incense stoves to improve air quality. With the function of purifying indoor air, eliminating odors, sterilizing and disinfecting, the aroma produced by incense can also refresh people's brains, clarify their thoughts, improve work efficiency, soothe tense emotions, relieve stress, and improve symptoms of insomnia and dizziness, etc.

However, the frequent usage of heating lamp heads of existing candle warmer lamps would cause high temperatures, resulting in the easy damage of heating lamp heads. Moreover, the lamp heads of the existing candle warmer lamps are fixedly connected to the lamp holders and cannot be replaced. Once the lamp head is damaged, the candle warmer lamp cannot be used, resulting in a shorter service life of the candle warmer lamp.

### SUMMARY

The present disclosure discloses a candle warmer lamp including a heating device, a base, and a supporting part; wherein the heating device is connected to the base through the supporting part, and the heating device is located above the base; the heating device includes a lamp holder and a heating lamp head, the lamp holder and the heating lamp head are detachably connected.

Further, the lamp holder is provides with a first clamping connector, and the heating lamp head is provides with a second clamping connector; and the first clamping connector and the second clamping connector are detachably connected.

Further, the lamp holder can also be provides with a third clamping connector; the heating lamp head is provides with a fourth clamping connector, and the third clamping connector is detachably connected with the fourth clamping connector.

Further, the first clamping connector includes a first guiding groove and a first locking groove, and the first locking groove is interconnected with the first guiding groove; when the second clamping connector is placed in the first guiding groove, the second clamping connector is able to slide into the first locking groove from the first guiding groove; the first locking groove is provides with a first stop edge, and the second clamping connector is provides with a second stop edge; when the second stop edge of the second clamping connector slides into the first locking groove from the first guiding groove, the first stop edge locks the second stop edge of the second clamping connector into the first locking groove.

Further, the third clamping connector includes a second guiding groove and a second locking groove, and the second guiding groove is interconnected with the second locking groove; when the fourth clamping connector is placed in the second guiding groove, the fourth clamping connector is able to slide into the second locking groove from the second guiding groove, and the second locking groove is provides with a third stop edge, and the fourth clamping connector is provides with a fourth stop edge; when the fourth stop edge of the fourth clamping connector slides into the second locking groove from the second guiding groove, the third stop edge locks the fourth stop edge of the fourth clamping connector into the second locking groove.

Further, the first locking groove is provides with a first conductive spring piece, and the second locking groove is provides with a second conductive spring piece; when the second clamping connector slides into the first locking groove from the first guiding groove, and the first stop edge locks the second stop edge of the second clamping connector in the first locking groove, the first conductive spring piece presses and locks the second clamping connector tightly in the first locking groove; when the fourth clamping connector is placed in the second guiding groove, and the fourth stop edge of the fourth clamping connector slides into the second locking groove from the second guiding groove, the second conductive spring piece presses and locks the fourth clamping connector tightly in the second locking groove.

Further, the first guiding groove and the second guiding groove are diagonal settings, the first locking groove and the second locking groove are diagonal settings.

Further, are diagonal settings includes a lamp cover, the lamp cover is connected with the first end of the supporting part through the first connecting assembly, and the base is connected with the second end of the supporting part through the second connecting assembly.

Further, the first end of the supporting part is provided with a fifth stop edge, the lampshade is sleeved at the first end of the supporting part, the first connecting assembly is connected with the first end of the supporting part, and the first connecting assembly together with the fifth stop edge clamp the lampshade tightly.

Further, the first connecting assembly includes a first nut, the first nut is connected with the first end of the supporting part; such that the first nut and the fifth stop edge clamp the lampshade tightly; a gasket is arranged between the first nut and the lampshade.

Further, the first connecting assembly also includes a fixing piece, the lamp holder is connected with one end of the fixing piece, the other end of the fixing piece is connected with the first end of the supporting part, and the fixing piece presses the first nut, the gasket, and the lampshade to the fifth stop edge tightly.

Further, the fixing piece is a hollow circular table with a narrow top and a wide bottom, the fixing piece forms an upper end surface located on the upper side of the fixing piece and a lower end surface located on the lower side of the fixing piece, the upper end surface is provides with internal threads, and the other end of the lower end surface is provides with a first fixing hole; the first end of the supporting part is provides with external threads, and the external threads on the first end of the supporting part are connected to the internal threads of the upper end surface, which is more convenient for the assembly of the candle warmer lamp; the lamp holder is provides with a second fixing hole, the second fixing hole is fixed with the first fixing hole by bolts, and this enables the connection between the lamp holder and the fixing piece.

Further, the bottom of the base is provided with a installation groove, and the base is provided with a first installation hole corresponding to the installation groove; the first installation hole is arranged corresponding to the second end of the supporting part, and the second connecting assembly includes a sixth stop edge and a second nut; the sixth stop edge is arranged on the second end of the supporting part, the supporting part passes through the first installation hole and is placed in the installation groove; the sixth stop edge is clamped on the base and screwed onto the second end of the supporting part through the second nut, so as to fix the base to the supporting part.

Further, the heating device also includes a power cord, with a supporting part being a hollow tube, a second installation hole corresponding to the installation groove is provided on the base; the power cord is arranged in the supporting part, and one end of the power cord is connected to the lamp holder; the other end of the power cord is connected to the base and passes through the second installation hole.

Further, the power cord is provides with a plug at an end, and the power cord is provides with a controller.

Further, the controller can be set with a power on/off key, a temperature increase/decrease key, and a heating timing key.

Further, the base is provides with a detachable non-slip layer, which covers the installation groove.

Further, the base is provides with a holding groove, the holding groove is configured to accommodate the incense candle.

The beneficial effect of the present disclosure is that a detachable connection structure is arranged between the lamp holder and the heating lamp head of the candle warmer lamp, which facilitates the replacement of the heating lamp head.

### BRIEF DESCRIPTION OF THE DRAWINGS

For the purpose of a clearer description of the embodiments in this application or technical solutions in prior art, below is a brief introduction of the attached drawings needed to be used in the description of the embodiments or prior art. Apparently, the attached drawings in the following description are only some embodiments indicated in the present application. For ordinary skill in the art, they may obtain other drawings according to these attached drawings without any innovative laboring.

The present disclosure will be further described with reference to the attached drawings and the embodiments hereunder.

FIG. 1 is a schematic diagram of an overall structure of a candle warmer lamp of the present disclosure.

FIG. 2 is an exploded view of the candle warmer lamp of FIG. 1.

FIG. 3 is an exploded view of a part of the candle warmer lamp of FIG. 1.

FIG. 4 is an exploded view of another part the candle warmer lamp of FIG. 1.

FIG. 5 is a schematic view of a lamp holder of the candle warmer lamp of FIG. 1.

FIG. 6 is another schematic view of the lamp holder of the candle warmer lamp of FIG. 5

FIG. 7 is a schematic view of a base of the candle warmer lamp of FIG. 5.

### DETAILED DESCRIPTION OF THE EMBODIMENTS

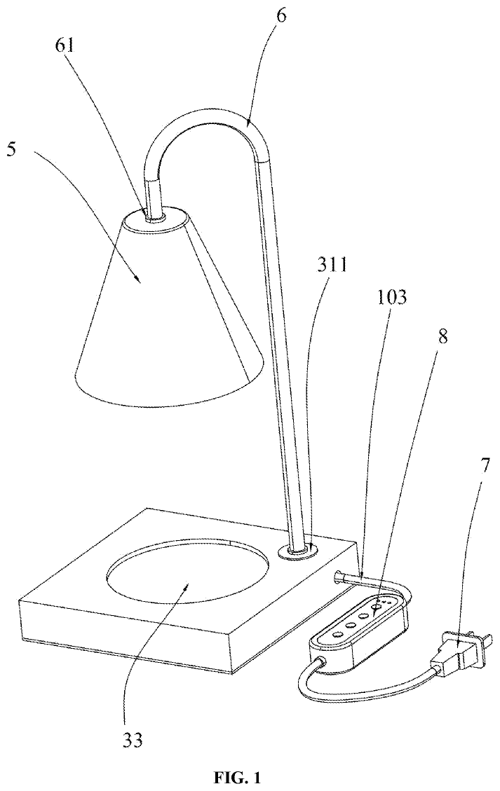

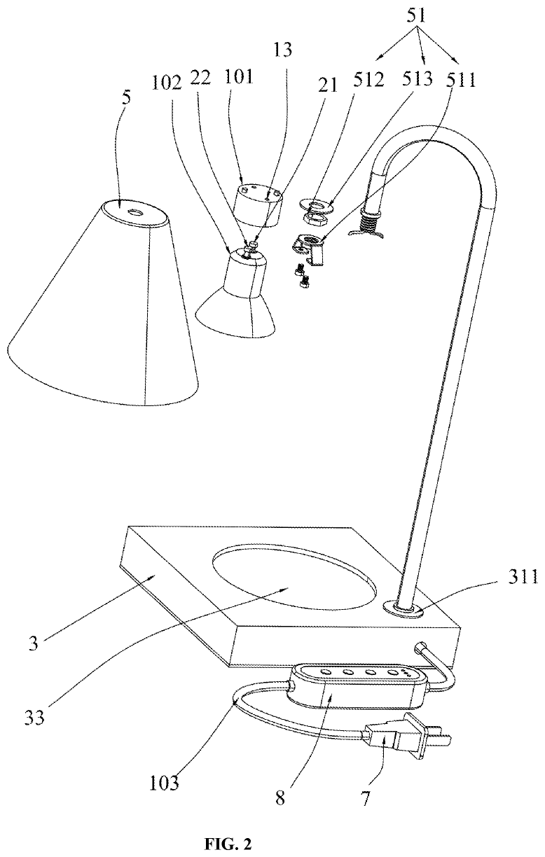

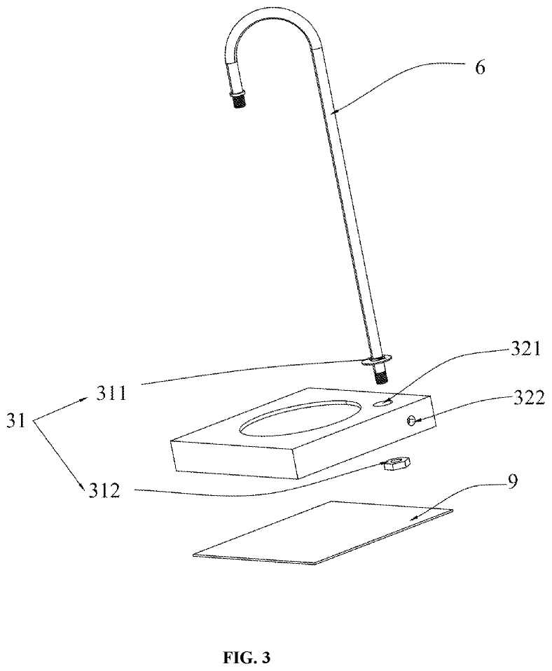

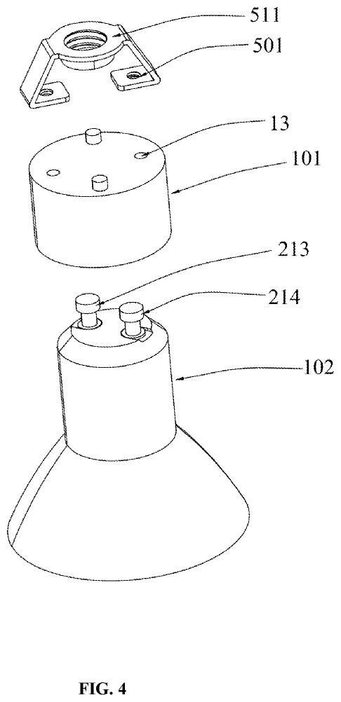

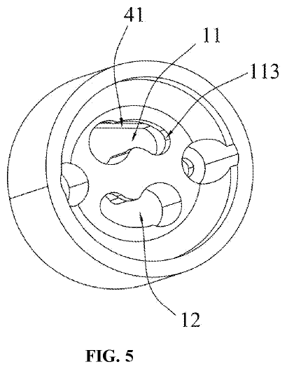

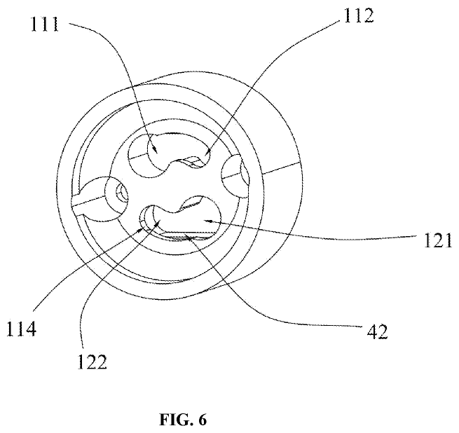

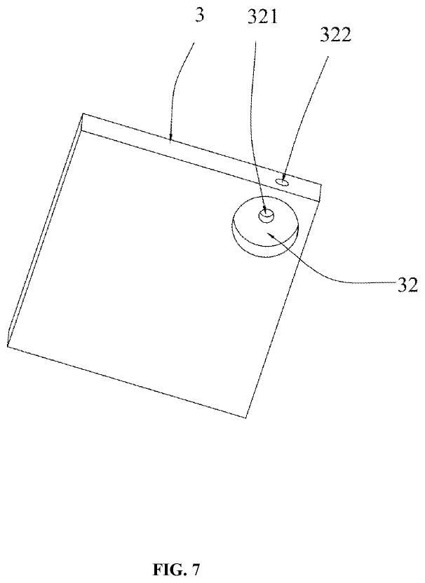

Referring to FIG. 1 to FIG. 7, a candle warmer lamp in this embodiment includes a heating device, a base 3, and a supporting part 6. The heating device is connected to the base 3 through the supporting part 6, and the heating device is located above the base. The heating device includes a lamp holder 101 and a heating lamp head 102, which are detachably connected. The lamp holder 101 is provides with a first clamping connector 11, and the heating lamp head 102 is provides with a second clamping connector 21. The first clamping connector 11 and the second clamping connector 21 are detachably connected. If the lamp head of the candle warmer lamp is damaged, the lamp holder 101 can also be provides with a third clamping connector 12. The heating lamp head 102 is provides with a fourth clamping connector 22, and the third clamping connector 12 is detachably connected with the fourth clamping connector 22. If the heating lamp head 102 is damaged, or the power line is in poor contact, it is convenient to install and replace the heating lamp head 102, so that the service life of the candle warmer lamp lasts longer. The base 3 is connected with the second end of the supporting part 6 through a second connecting assembly 31. The candle warmer lamp can also include a lampshade 5, which can converge light, so that the heat of the heating lamp head 102 can be aggregated to the incense candle, resulting in the faster melting of the incense candle and diffusing of its fragrance. The lampshade 5 is connected with the first end of the supporting part 6 through a first connecting assembly 51. The first end of the supporting part 6 is provided with a fifth stop edge 61. The lampshade 5 is set at the first end of the supporting part 6, and the first connecting assembly 51 is connected with the first end of the supporting part 6. The first connecting assembly 51 and the fifth stop edge 61 clamp the lampshade 5 tightly.

In this embodiment, the first clamping connector 11 includes a first guiding groove 111 and a first locking groove 112. The first locking groove 112 is interconnected with the first guiding groove 111. When the second clamping connector 21 is placed in the first guiding groove 111, it can slide into the first locking groove 112 from the first guiding groove 111. The first locking groove 112 is provides with a first stop edge 113, and the second clamping connector 21 is provides with a second stop edge 213. When the second stop edge 213 of the second clamping connector 21 slides into the first locking groove 112 from the first guiding groove 111, the first stop edge 113 locks the second stop edge 213 of the second clamping connector 21 into the first locking groove 112. The third clamping connector 12 includes a second guiding groove 121 and a second locking groove 122, and the second guiding groove 121 is interconnected with the second locking groove 122. When the fourth clamping connector 22 is placed in the second guiding groove 121, it can slide into the second locking groove 122 from the second guiding groove 121. The second locking groove 122 is provides with a third stop edge 114, and the fourth clamping connector 22 is provides with a fourth stop edge 214. When the fourth stop edge 214 of the fourth clamping connector 22 slides into the second locking groove 122 from the second guiding groove 121, the third stop edge 114 locks the fourth stop edge 214 of the fourth clamping connector 22 into the second locking groove 122. This embodiment of sliding connection enables users to replace the heating lamp head conveniently.

In this embodiment, the first locking groove 112 is provides with a first conductive spring piece 41, and the second locking groove 122 is provides with a second conductive spring piece 42. When the second clamping connector 21 slides into the first locking groove 112 from the first guiding groove 111, and the first stop edge 113 locks the second stop edge 213 of the second clamping connector 21 in the first locking groove 112, the first conductive spring piece 41 presses and locks the second clamping connector 21 tightly in the first locking groove 112. When the fourth clamping connector 22 is placed in the second guiding groove 121, and the fourth stop edge 214 of the fourth clamping connector 22 slides into the second locking groove 122 from the second guiding groove 121, the second conductive spring piece 42 presses and locks the fourth clamping connector 22 tightly in the second locking groove 122. The first conductive spring piece 41 and the second conductive spring piece 42 enable the connection of the first clamping connector 11 and the third clamping connector 12, as well as the second clamping connector 21 and the fourth clamping connector 22, so that the heating lamp head can be energized. Of course, the first and the third clamping connectors also require a conductive metal structure. The first clamping connector 11 and the third clamping connector 12 are mirror settings, while the first guiding groove 111 and the second guiding groove 121 are diagonal settings, so are the first locking groove 112 and the second locking groove 122. The second clamping connector 21 and the fourth clamping connector 22 are simultaneously placed in the first guiding groove 111 and the second guiding groove 121. They twist clockwise or counterclockwise to enter the first locking groove 112 and the second locking groove 122, which are tightly locked and clamped. This clamping structure is simpler, and the installation of the heating lamp head and the lamp holder 101 is more convenient and stable.

In this embodiment, the first connecting assembly 51 includes a first nut 512, which is connected with the first end of the supporting part 6. This enables the first nut 512 and the fifth stop edge 61 to clamp the lampshade 5 tightly. A gasket 513 is arranged between the first nut 512 and the lampshade 5. The first connecting assembly 51 also includes a fixing piece 511. The lamp holder 101 is connected with one end of the fixing piece 511, and the other end of the fixing piece 511 is connected with the first end of the supporting piece 6. The fixing piece 511 presses the first nut 512, the gasket 513, and the lampshade 5 tightly to the fifth stop edge 61. Here, the fixing of the first end of the supporting piece 6 to the base is not limited to the use of nuts, and direct welding can be utilized.

In this embodiment, the fixing piece 511 is a hollow circular table with a narrow top and a wide bottom, forming an upper end surface located on the upper side of the fixing piece and a lower end surface located on the lower side of the fixing piece. The upper end surface is provides with internal threads, and the other end of the lower end surface is provides with a first fixing hole 501. The first end of the supporting part 6 is provides with external threads, and the external threads on the first end of the supporting part 6 are connected to the internal threads of the upper end surface, which is more convenient for the assembly of the candle warmer lamp. The lamp holder 101 is provides with a second fixing hole 13, which is fixed with the first fixing hole 501 by bolts. This bolt setting enables and eases the connection between the lamp holder 101 and the fixing piece 511. The setting of the fixing piece 511 facilitates the disassembly and assembly between the lamp holder and the supporting piece.

In this embodiment, the bottom of the base 3 is provided with a installation groove 32, and the base 3 is provided with a first installation hole 321 corresponding to the installation groove 32. The first installation hole 321 is arranged corresponding to the second end of the supporting part 6. The second connecting assembly 31 includes a sixth stop edge 311 and a second nut 312. The sixth stop edge 311 is arranged on the second end of the supporting part 6, which passes through the first installation hole 321 and is placed in the installation groove 32. Also, the sixth stop edge 311 is clamped on the base 3 and screwed onto the second end of the supporting part 6 through the second nut 312, thus to fix the base 3 to the supporting part 6. The installation groove 32 is convenient for the disassembly and assembly of the power cord. The base 3 is also provides with a holding groove 33, which is used to accommodate the incense candle. In this way, the incense candle is much stable and safe.

In this embodiment, the heating device also includes a power cord 103, with a supporting part 6 being a hollow tube, and a second installation hole 322 corresponding to the installation groove 32 on the base 3. The power cord 103 is arranged in the supporting part 6, and one end of the power cord 103 is connected to the lamp holder 101. The other end of the power cord 103 is connected to the base 3 and passes through the second installation hole 322. The arrangement of the second installation hole 322 is mainly for the convenience of the power cord installation, making the power cord fitly organized and the candle warmer lamp more beautiful and tidy.

In this embodiment, the power cord 103 is provides with a plug 7 at the end, and the power cord 103 is provides with a controller 8. The controller 8 can be set with a power on/off key, a temperature increase/decrease key, and a heating timing key which can be designed to be 2 hours, 4 hours, and 8 hours. The controller 8 is compact in structure and reasonable in layout with only one control button for switching and timing control. The candle warmer lamp of switching and timing can effectively assist in reminding the working status of the electronic incense stove. A thermometer with a magnifying glass is also arranged to make it easy to master the temperature of the electronic incense stove.

In this embodiment, the base 3 is provides with a detachable waterproof layer 9, which covers the installation groove 32. If the base 3 is supported by wood, it can prevent water from penetrating the base.

The overall structure of the candle warmer lamp of the present disclosure is simple, compact, and reasonably arranged. With only one clamping structure, damaged heating lamp heads can be replaced, and the service life of the candle warmer lamp can be extended. The overall design is convenient for disassembly and assembly, maintenance, and control buttons are also set for switching control and timing control. The candle warmer lamp of switching and timing can also be set to assist in reminding the current working status of the candle warmer lamp.

What is claimed is:

1. A candle warmer lamp, comprises a heating device, a base (3), and a supporting part (6); wherein the heating device is connected to the base (3) through the supporting part (6), and the heating device is located above the base; the heating device comprises a lamp holder (101) and a heating lamp head (102), the lamp holder (101) and the heating lamp head (102) are detachably connected.

2. The candle warmer lamp according to claim 1, wherein the lamp holder (101) is provides with a first clamping connector (11), and the heating lamp head (102) is provides with a second clamping connector (21); and the first clamping connector (11) and the second clamping connector (21) are detachably connected.

3. The candle warmer lamp as claimed in claim 2, wherein the lamp holder (101) is provides with a third clamping connector (12); the heating lamp head (102) is provides with a fourth clamping connector (22), and the third clamping connector (12) is detachably connected with the fourth clamping connector (22).

4. The candle warmer lamp as claimed in claim 3, wherein the first clamping connector (11) comprises a first guiding groove (111) and a first locking groove (112), and the first locking groove (112) is interconnected with the first guiding groove (111); when the second clamping connector (21) is placed in the first guiding groove (111), the second clamping connector (21) is able to slide into the first locking groove (112) from the first guiding groove (111); the first locking groove (112) is provides with a first stop edge (113), and the second clamping connector (21) is provides with a second stop edge (213); when the second stop edge (213) of the second clamping connector (21) slides into the first locking groove (112) from the first guiding groove (111), the first stop edge (113) locks the second stop edge (213) of the second clamping connector (21) into the first locking groove (112).

5. The candle warmer lamp as claimed in claim 4, wherein the third clamping connector (12) comprises a second guiding groove (121) and a second locking groove (122), and the second guiding groove (121) is interconnected with the second locking groove (122); when the fourth clamping connector (22) is placed in the second guiding groove (121), the fourth clamping connector (22) is able to slide into the second locking groove (122) from the second guiding groove (121), and the second locking groove (122) is provides with a third stop edge (114), and the fourth clamping connector (22) is provides with a fourth stop edge (214); when the fourth stop edge (214) of the fourth clamping connector (22) slides into the second locking groove (122) from the second guiding groove (121), the third stop edge (114) locks the fourth stop edge (214) of the fourth clamping connector (22) into the second locking groove (122).

6. The candle warmer lamp as claimed in claim 5, wherein the first locking groove (112) is provides with a first conductive spring piece (41), and the second locking groove (122) is provides with a second conductive spring piece (42); when the second clamping connector (21) slides into the first locking groove (112) from the first guiding groove (111), and the first stop edge (113) locks the second stop edge (213) of the second clamping connector (21) in the first locking groove (112), the first conductive spring piece (41) presses and locks the second clamping connector (21) tightly in the first locking groove (112); when the fourth clamping connector (22) is placed in the second guiding groove (121), and the fourth stop edge (214) of the fourth clamping connector (22) slides into the second locking groove (122) from the second guiding groove (121), the second conductive spring piece (42) presses and locks the fourth clamping connector (22) tightly in the second locking groove (122).

7. The candle warmer lamp as claimed in claim 6, wherein the first guiding groove (111) and the second guiding groove (121) are diagonal settings, the first locking groove (112) and the second locking groove (122) are diagonal settings.

8. The candle warmer lamp as claimed in claim 1, wherein the candle warmer lamp further comprises a lamp cover (5), the lamp cover (5) is connected with the first end of the supporting part (6) through the first connecting assembly (51), and the base (3) is connected with the second end of the supporting part (6) through the second connecting assembly (31).

9. The candle warmer lamp as claimed in claim 8, wherein the first end of the supporting part (6) is provided with a fifth stop edge (61), the lampshade (5) is sleeved at the first end of the supporting part (6), the first connecting assembly (51) is connected with the first end of the supporting part (6), and the first connecting assembly (51) together with the fifth stop edge (61) clamp the lampshade (5) tightly.

10. The candle warmer lamp as claimed in claim 9, wherein the first connecting assembly (51) comprises a first nut (512), the first nut (512) is connected with the first end of the supporting part (6) such that the first nut (512) and the fifth stop edge (61) clamp the lampshade (5) tightly; a gasket (513) is arranged between the first nut (512) and the lampshade (5).

11. The candle warmer lamp as claimed in claim 10, wherein the first connecting assembly (51) also comprises a fixing piece (511), the lamp holder (101) is connected with one end of the fixing piece (511), the other end of the fixing piece (511) is connected with the first end of the supporting part (6), and the fixing piece (511) presses the first nut (512), the gasket (513), and the lampshade (5) to the fifth stop edge (61) tightly.

12. The candle warmer lamp as claimed in claim 11, wherein the fixing piece (511) is a hollow circular table with a narrow top and a wide bottom, the fixing piece (511) forms an upper end surface located on an upper side of the fixing piece and a lower end surface located on a lower side of the fixing piece, the upper end surface is provides with internal threads, and the other end of the lower end surface is provides with a first fixing hole (501); the first end of the supporting part (6) is provides with external threads, and the external threads on the first end of the supporting part (6) are connected to the internal threads of the upper end surface; the lamp holder (101) is provides with a second fixing hole (13), the second fixing hole (13) is fixed with the first fixing hole (501) by a bolt so as to connect the lamp holder (101) and the fixing piece (511).

13. The candle warmer lamp according to claim 8, wherein the bottom of the base (3) is provided with a installation groove (32), and the base (3) is provided with a first installation hole (321) corresponding to the installation groove (32); the first installation hole (321) is arranged corresponding to the second end of the supporting part (6), and the second connecting assembly (31) comprises a sixth stop edge (311) and a second nut (312); the sixth stop edge (311) is arranged on the second end of the supporting part (6), the supporting part (6) passes through the first installation hole (321) and is placed in the installation groove (32); the sixth stop edge (311) is clamped on the base (3) and screwed onto the second end of the supporting part (6) through the second nut (312), so as to fix the base (3) to the supporting part (6).

14. The candle warmer lamp as claimed in claim 13, wherein the heating device also comprises a power cord (103), the supporting part (6) is a hollow tube, and a second installation hole (322) corresponding to the installation groove (32) is provided on the base (3); the power cord (103) is arranged in the supporting part (6), one end of the power cord (103) is connected to the lamp holder (101), the other end of the power cord (103) is connected to the base (3) and passes through the second installation hole (322).

15. The candle warmer lamp as claimed in claim 14, wherein the power cord (103) is provides with a plug (7) at an end, and the power cord (103) is provides with a controller (8).

16. The candle warmer lamp as claimed in claim 15, wherein the controller (8) includes a power on/off key, a temperature increase/decrease key, and a heating timing key.

17. The candle warmer lamp as claimed in claim 13, wherein the base (3) is provides with a detachable non-slip layer (9), and the detachable waterproof layer (9) covers the installation groove (32).

18. The candle warmer lamp as claimed in claim 1, wherein the base (3) is provides with a holding groove (33), the holding groove (33) is configured to accommodate the incense candle.
